(19) United States
(12) Patent Application Publication
(10) Pub. No.: US 2026/0314934 A1
(43) Pub. Date: Oct. 15, 2026

(54) PROBE SET FOR MONITORING GAS

(71) Applicant: Neola Medical AB, Lund (SE)

(72) Inventors: Rasmus GRÖNLUND, Dalby (SE); Sara BERGSTEN, Lund (SE); Martin HANSSON, Södra Sandby (SE)

(73) Assignee: Neola Medical AB, Lund (SE)

(21) Appl. No.: 19/484,512

(22) PCT Filed: May 14, 2024
(86) PCT No.: PCT/EP2024/063292

§ 371 Date: Nov. 14, 2025

(30) Foreign Application Priority Data
May 14, 2023 (SE) 2350584-5

(51) Int. Cl. A61B 5/00; A61B 5/08

(52) U.S. Cl. CPC A61B 5/0075; A61B 5/08; A61B 5/688

(57) ABSTRACT

A probe set (1) comprises a light emitter probe and a light detector probe adapted to be used for monitoring gas in a cavity of a subject. The emitter probe comprises an emitter head (2) connected to a first connector interface (3) and the detector probe comprises a detector head (4) connected to a second connector interface (5). The probe set (1) further comprises a plastic tab (6) having light attenuation properties, wherein the emitter head (2) is arranged on one side of the tab and the detector head (4) is arranged opposite the emitter head (2) on the other side of the plastic tab (6) such that a light emitting area of the emitter head (2) and a light collecting area of the detector head (4) faces each other with the plastic tab (6) having light attenuation properties in-between.

### TECHNICAL FIELD

The present invention relates to a probe set comprising a light emitter probe and a light detector probe adapted to be used for monitoring gas in a cavity of a subject. The emitter probe comprises an emitter head connected to a first connector interface and the detector probe comprises a detector head connected to a second connector interface.

### BACKGROUND

A probe set adapted to be used for monitoring gas in a cavity generally consists of an emitter probe and a detector probe. The purpose of the emitter probe is to guide light to a subject, generally a patient, and to distribute the light evenly over a small area of the subject's skin. The detector probe is used to collect light from a small area of the subject's skin and detect the light, i.e. convert the collected light into an electrical signal, and deliver the signal to a control unit.

Gas in scattering media absorption spectroscopy is a novel optical technology employing the gas absorption lines in the near-infrared region. It has a potential use in the medical setting as a monitoring and diagnostic tool by detecting molecular oxygen gas and water vapor absorption within gas pockets and thus may be a useful adjunct in respiratory monitoring. It has potential advantages over other monitoring devices currently used in clinical practice. It is a non-invasive, continuous, non-ionizing technology and provides unique information about molecular oxygen content inside the lungs. It may have a future role in instant detection of pulmonary complications and optimizing respiratory management of neonates in different clinical scenarios such as detecting lung collapses, monitoring cardiorespiratory transition in the delivery room and optimizing endotracheal tube positioning.

Prior to use of a medical device based on gas absorption spectroscopy, the gas absorption by the gas inside the device needs to be quantified and subtracted from the gas absorption measured in the cavity of a subject, a kind of offset measurement and adjustment. Fluctuating air humidity causes differences in base values, making day-to-day adjustments necessary. This adjustment is performed by measuring the spectroscopic signal with the emitter and detector probes connected together without any air gap between them. In this setup, the light needs to be attenuated to avoid saturating the detector.

### SUMMARY

It is an object of the present invention to provide a probe set that facilitates the use and handling of the set.

According to a first aspect of the present disclosure a probe set comprises a light emitter probe and a light detector probe adapted to be used for monitoring gas in a cavity of a subject. The emitter probe comprises an emitter head connected to a first connector interface and the detector probe comprises a detector head connected to a second connector interface. The probe set further comprises a plastic tab having light attenuation properties. The emitter head is arranged on one side of the tab and the detector head is arranged opposite the emitter head on the other side of the plastic tab such that a light emitting area of the emitter head and a light collecting area of the detector head faces each other with the plastic tab having light attenuation properties in-between. A pre-requisite is that no gas is allowed between the heads at this stage.

This set up facilitates offset measurement and adjustment, since only the interfaces of the probes need to be connected to a control unit and the light attenuation properties provides for a sufficient level of light attenuation or light suppression. In other words, the probes need not be removed from the plastic tab until it is time to arrange them on the subject for monitoring. The plastic tab should have a certain degree of mechanical stability, i.e., a rigid material, such that it does not easily fold, for instance, somewhere between 0.3 mm and 0.5 mm thick. Also, the plastic tab is preferably of medical grade. An option is to use a rigid PET sheet.

One example of a plastic tab having light attenuation properties is a plastic tab with a light attenuation film to provide the light attenuation properties. A further example of a plastic tab having light attenuation properties is a plastic tab made of a material that has known light attenuation properties such that no additional film is required. Yet another example is to have the plastic tab coated with a material that has light attenuation properties, for instance, with paint or a powder coating.

According to an aspect of the present disclosure the emitter head and detector head are attached with adhesive tape to the plastic tab. This provides for an easy and cost-efficient assembly of the probe set. Properly carried out, attaching the heads with an adhesive tape ensures that no air or other gas is trapped between the heads.

According to a further aspect of the present disclosure at least one edge of the plastic tab is straight and the adhesive tape is folded about said edge such that one single adhesive tape is adapted to hold both the emitter head and the detector head. Again, this provides for an easy assembly of the probe set and is hence cost efficient.

For further efficiency, the adhesive tape contains the light attenuation film according to a preferred aspect of the present disclosure.

Hence, according to yet another aspect of the present disclosure, the light attenuation film is part of an adhesive tape strip attached at one end on one side of the plastic tab and the adhesive tape is folded around the plastic tab such that the emitter and detector heads are attached to the plastic tab facing each other with the plastic tab and one layer of the adhesive tape in-between.

According to another aspect of the present disclosure the width of the adhesive tape strip at the end attached to the plastic tab is wider than the remaining adhesive tape strip. This facilitates a centered placement of the adhesive tape on the plastic tab.

According to yet another aspect of the present disclosure a light emitting area of the emitter head has an adhesive layer with a first liner and a light collecting area on the detector head has an adhesive layer with a second liner. Preferably, the shape of at least the second liner corresponds to the shape of the plastic tab. This facilitates arranging the detector head in the correct position. To further facilitate arranging the second liner, the size of the second liner corresponds to the size of the plastic tab.

According to another aspect of the present disclosure, the light collecting area of the detector head is a photodiode. Hence the connection between the detector head and the second connector interface could be made using a regular electric cable.

According to a further aspect of the present disclosure the probe set comprises a probe set inlay with an optical fiber and/or wire holding element and wherein the plastic tab is attached to the probe set inlay. Thus, the entire probe set is allowed to be handled as a single unit.

Preferably, the probe sets are each packed separately in a standard bag, for instance a Tyvek bag, where one side is clear plastic, and the other side is made of paper-like material. Preferably, two holes are punched on the upper edge for hanging the probe set on a hook on the side of the control unit.

The probe set inlay and the plastic tab is made in one piece according to an aspect of the present disclosure. Thus, the inlay could be produced by proper punching out of a plastic film.

As previously mentioned concerning the plastic tab, the inlay should have a certain mechanical stability being made of a plastic film that typically is between 0.3 mm and 0.5 mm thick, such as for example a rigid PET sheet.

According to a preferred aspect of the present disclosure the holding element is a punched out part of the inlay. Thus, cables/optical fiber could be held with this element that is part of the inlay.

Also, according to yet another aspect of the present disclosure, the inlay has a hole in an upper part and the plastic tab is arranged at a lower part of the inlay. The hole could be used to hang the probe set on a hook and having the plastic tab on the lower part could facilitate the handling of the probe heads.

According to an alternative aspect of the present disclosure a method of assembling a probe set comprising a light emitter probe and a light detector probe that are adapted to be used for monitoring gas in a cavity of a subject is provided. The emitter probe comprises an emitter head connected to a first connector interface and the detector probe comprises a detector head connected to a second connector interface. The method comprises attaching the light emitter head and the light detector head to a plastic tab with one head on each side of the plastic tab and facing each other with the plastic tab in-between the two heads, and wherein the plastic tab has light attenuation properties.

The method according to a second aspect of the present disclosure comprises providing a light attenuation film to provide the light attenuation properties. Attaching an end of a tape with the light attenuation film to the plastic tab and folding it around the plastic tab such that the emitter head and the detector head are attached to the plastic tab with the same tape.

Light attenuation properties are properties which attenuate a light beam, i.e., reduces its optical power/intensity. The amount of attenuation, the overall transmission loss, is often quantified as an optical density and/or absorbance. For example, the light attenuation properties could have an approximately constant degree of attenuation (loss) in a substantial wavelength range.

It should be emphasized that the term “comprises/comprising” when used in this specification is taken to specify the presence of stated features, integers, steps or components but does not preclude the presence or addition of one or more other features, integers, steps, components or groups thereof.

Further features of, and advantages with, the present invention will become apparent when studying the appended claims and the following description. The skilled person realizes that different features of the present invention may be combined to create embodiments other than those described in the following, without departing from the scope of the present invention.

### BRIEF DESCRIPTION OF THE DRAWINGS

The above, as well as additional objects, features and advantages of the present invention, will be better understood through the following illustrative and non-limiting detailed description of exemplary embodiments of the present invention, wherein:

FIGS. 1 and 2 show a front side of a probe set according to an embodiment according to the present disclosure.

FIGS. 3 and 4 show a back side of a probe set according to an embodiment according to the present disclosure.

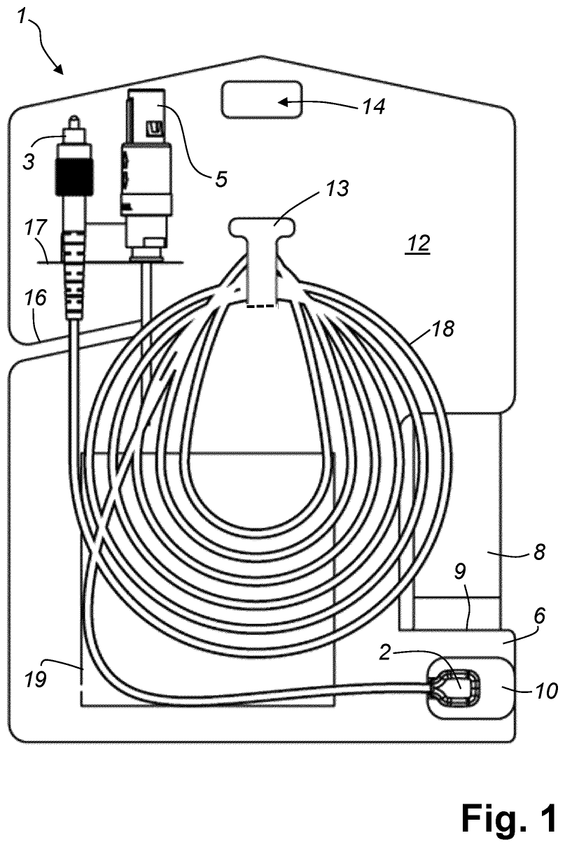

### DETAILED DESCRIPTION OF EXAMPLE EMBODIMENTS OF THE INVENTION

Specific embodiments of the invention now will be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

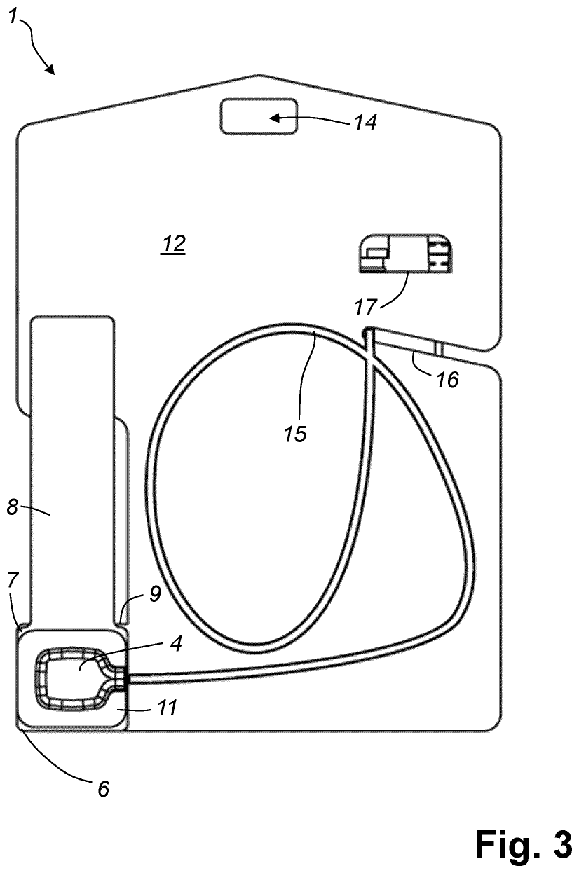

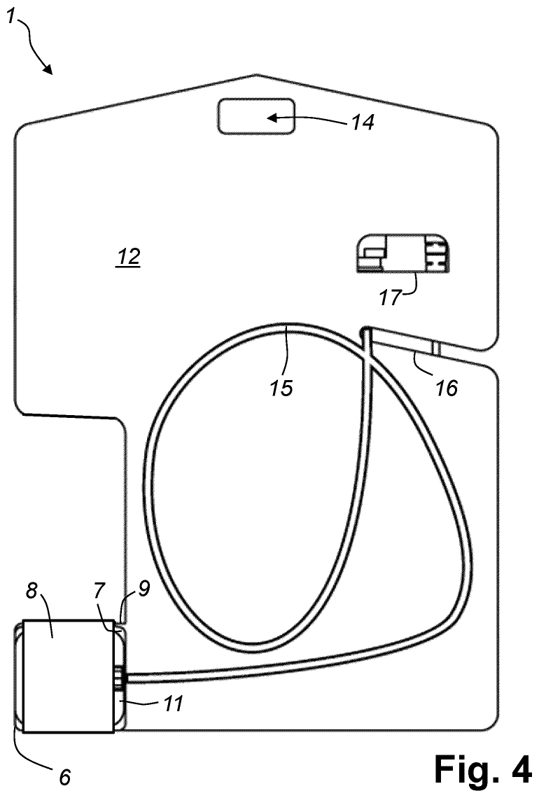

With reference to the figures, the probe set 1 comprises a light emitter probe and a light detector probe that are adapted to be used for monitoring gas in a cavity of a subject. The emitter probe comprises an emitter head 2 connected to a first connector interface 3 and the detector probe comprises a detector head 4 connected to a second connector interface 5. The probe set 1 further comprises a plastic tab 6 having light attenuation properties. The light attenuation properties may come from a light attenuation film 7, see FIGS. 3 and 4. The emitter head 2 is arranged on one side of the tab and the detector head 4 is arranged opposite the emitter head 2 on the other side of the plastic tab 6 such that a light emitting area of the emitter head 2 and a light collecting area of the detector head 4 faces each other with the light attenuation film 7 in-between.

Alternatively, in some examples may a light attenuation coating, such as a paint or powder coating, be applied to the plastic tab 6 instead of a light attenuation film 7. In other examples may the plastic tab 6 be made of a material that has known light attenuation properties, such as the plastic tab 6 may be made of a colored plastic having light attenuation properties.

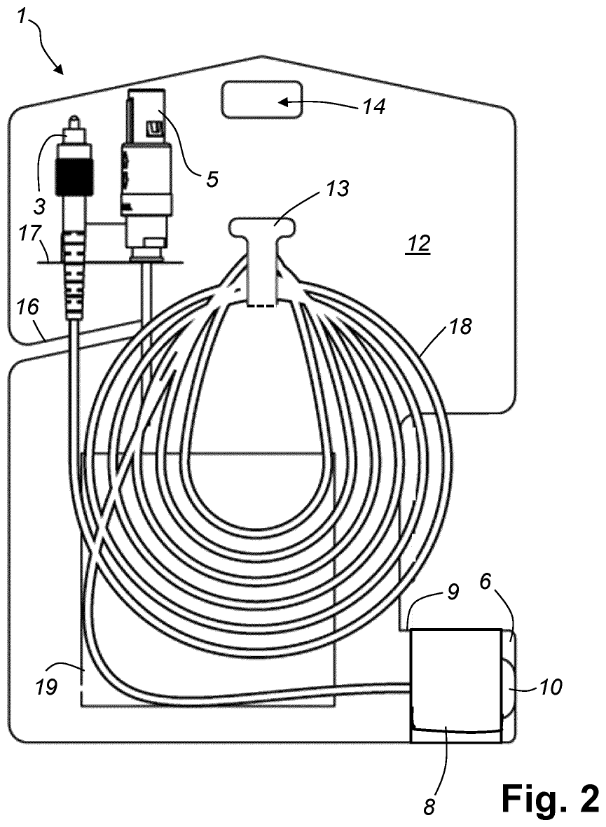

As further can be seen in FIGS. 2 and 4, the emitter head 2 and detector head 4 are attached with adhesive tape 8 to the plastic tab 6. Also, one edge 9 of the plastic tab 6 is straight and the adhesive tape is folded about said edge 9 such that one single adhesive tape 8 is adapted to hold both the emitter head 2 and the detector head 4.

Alternatively, and/or additionally, to using an adhesive tape, other fasteners for attaching the emitter head 2 and detector head 4 to the plastic tab 6 may be used, such as velcro, metallic wires or strips (such as a twist tie), straps, clamps or clips. Alternatively, the plastic tab 6 may be, for example, molded into mechanical holders for holding the emitter head 2 and detector head 4 or be cut and folded into mechanical members holding the emitter head 2 and detector head 4 in place.

In the shown embodiment, the adhesive tape 8 contains the light attenuation film 7 and which is part of an adhesive tape strip 8 attached at one end to one side of the plastic tab 6 and wherein the adhesive tape 8 is folded around the plastic tab 6 such that the emitter and detector heads 2, 4 are attached to the plastic tab 6 facing each other with the plastic tab 6 and one layer of the adhesive tape 8 in-between.

Also, the width of the adhesive tape strip 8 at the end attached to the plastic tab is wider than the remaining adhesive tape strip 8. The tape consists according to one embodiment of 2 layers, one double sided medical grade adhesive and a light attenuation film. The wider part of the tape is placed between the probe heads 2, 4 and the strip 8 is used to strap the probe heads 2, 4 together. As the strip 8 also contains the light attenuation film, this will be helpful in suppressing background light when performing the offset measurement and adjustment.

A light emitting area of the emitter head 2 has an adhesive layer with a first liner 10 and a light collecting area on the detector head 4 has an adhesive layer with a second liner 11. The shape of at least the second liner 11 corresponds to the shape of the plastic tab 6. The probe holder tape 8 is placed on the tab in a lower corner, then the emitter and detector heads 2, 4 are placed on either side of the plastic tab 6, facing each other. The liner 11 of the detector head 4 is almost as large as the plastic tab 6 for easy centering. The emitter head 2 is to be attached with the liner 10 edge along the outer edge of the plastic tab 6, with the head 2 roughly centered vertically. The probe holder tape 8 is then strapped around the inlay corner piece, i.e., the plastic tab 6, and the probe heads 2, 4, keeping them in place.

As also can be seen the probe set 1 further comprises a probe set inlay 12 with an optical fiber and/or wire holding element 13 and wherein the plastic tab 6 is attached to the probe set inlay 12. In the shown embodiment the probe set inlay 12 and the plastic tab 6 is made in one piece.

A detector probe cable 15 is arranged on the back side of the inlay 12, the cable 15 is drawn through a slit 16 on one side of the inlay and the connector 5 is then attached to the inlay 12 by a bended punched out part 17 above the slit 16 and attaching the cable end in a slot.

The emitter probe optical fiber 18 is wrapped to form a loop of several laps. The long-term bend radius for the fiber is according to one embodiment 4.7 cm, so the loop has to have a diameter of at least 9.4 cm. All fiber, except the two laps closest to the connector 3 are attached to the inlay by bending the T-shaped plastic piece 13 out, placing the fiber 18 on top of it and then bending the plastic back so that the T-shape stays in the slot. The last laps of the fiber 18 are then wrapped into a circle and the connector 3 is attached to the plastic 17 next to the detector connector.

All probe sets are produced to be identical for simplifying usage. Each probe set comes with a set 19 of stickers for placing on the cables/fibers. The stickers are preferably labeled Dx (from latin: Dexter=right) and Sin (from latin: Sinister=left). For probes to be used on the subject's right side, the Dx stickers are used and for the subject's left side, Sin stickers are used. There are preferably four of each sticker, to allow placing stickers both close to the connector 3, 5 and close to the probe head 2, 4.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. For instance, instead of having an emitter probe optical fiber coupled to the emitter head for emitting light, the light emitted could be that produced by an LED arranged in the emitter head and thus the fiber replaced by an electric cable.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

1. A probe set (1) comprising a light emitter probe and a light detector probe adapted to be used for monitoring gas in a cavity of a subject, the emitter probe comprises an emitter head (2) connected to a first connector interface (3) and the detector probe comprises a detector head (4) connected to a second connector interface (5), characterized in that the probe set (1) further comprises a plastic tab (6) having light attenuation properties, wherein the emitter head (2) is arranged on one side of the tab and the detector head (4) is arranged opposite the emitter head (2) on the other side of the plastic tab (6) such that a light emitting area of the emitter head (2) and a light collecting area of the detector head (4) faces each other with the plastic tab (6) having light attenuation properties in-between.

2. The probe set (1) according to claim 1, where the light attenuation properties are provided by a light attenuation film (7).

3. The probe set (1) according to claim 1, wherein the emitter head (2) and detector head (4) are attached with adhesive tape (8) to the plastic tab (6).

4. The probe set (1) according to claim 3, wherein at least one edge (9) of the plastic tab (6) is straight and that the adhesive tape is folded about said edge (9) such that one single adhesive tape (8) is adapted to hold both the emitter head (2) and the detector head (4).

5. The probe set (1) according to claim, to 4, wherein the adhesive tape (8) contains the light attenuation film (7).

6. The probe set (1) according to claim 2, wherein the light attenuation film (7) is part of an adhesive tape strip (8) attached at one end to one side of the plastic tab (6) and wherein the adhesive tape (8) is folded around the plastic tab (6) such that the emitter and detector heads (2, 4) are attached to the plastic tab (6) facing each other with the plastic tab (6) and one layer of the adhesive tape (8) in-between.

7. The probe set (1) according to claim 6, wherein the width of the adhesive tape strip (8) at the end attached to the plastic tab is wider than the remaining adhesive tape strip (8).

8. The probe set (1) according to claim 1, wherein a light emitting area of the emitter head (2) has an adhesive layer with a first liner (10) and a light collecting area on the detector head (4) has an adhesive layer with a second liner (11), wherein the shape of at least the second liner corresponds to the shape of the plastic tab (6).

9. The probe set (1) according to claim 8, wherein the size of the second liner corresponds to the size of the plastic tab (6).

10. The probe set (1) according to claim 1, claims, wherein the light collecting area of the detector head (4) is a photodiode.

11. The probe set (1) according to claim 1, claims, further comprising a probe set inlay (12) with an optical fiber and/or wire holding element (13) and wherein the plastic tab (6) is attached to the probe set inlay (12).

12. The probe set (1) according to claim 11, wherein the probe set inlay (12) and the plastic tab (6) is made in one piece.

13. The probe set (1) according to claim 11, wherein the holding element (13) is a punched out part of the inlay (12).

14. The probe set (1) according to claim 11, wherein the inlay (12) has a hole (14) in an upper part and wherein the plastic tab (6) is arranged at a lower part of the inlay (12).

15. A method of assembling a probe set (1) comprising a light emitter probe and a light detector probe adapted to be used for monitoring gas in a cavity of a subject, the emitter probe comprising an emitter head (2) connected to a first connector interface (3) and the detector probe comprises a detector head (4) connected to a second connector interface (5), comprising attaching the light emitter head (2) and the light detector head (4) to a plastic tab (6) with one head (2, 4) on each side of the plastic tab (6) and facing each other with the plastic tab (6) in-between the two heads (2, 4), and wherein the plastic tab (6) has light attenuation properties.

16. The method of claim 15, where the light attenuation properties are provided by a light attenuation film (7) and the method comprising attaching an end of a tape (8) with the light attenuation film (7) to the plastic tab (6) and folding it around the plastic tab (6) such that the emitter head (2) and the detector head (4) are attached to the plastic tab (6) with the same tape (8).
